(19) United States
(12) Patent Application Publication
(10) Pub. No.: US 2003/0143178 A1
(43) Pub. Date: Jul. 31, 2003

(54) Pre-shampoo conditioning composition

(75) Inventors: Natsumi Komure, Ashiya-shi (JP); Miwa Nagao, Nishinomiya-shi (JP); Aiko Takabatake, Kobe (JP); Bruce Russell Cox, Clark, NJ (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/375,203

(22) Filed: Feb. 27, 2003

Related U.S. Application Data
(63) Continuation of application No. PCT/US00/24018, filed on Aug. 31, 2000.

(51) Int. Cl. A61K 7/06; A61K 7/11

(52) U.S. Cl. 424/70.12

(57) ABSTRACT

Disclosed is a hair conditioning composition for applying to the dry hair prior to washing the hair, comprising by weight: (a) from about 30% to about 99.9% of a conditioning oil which is in liquid form at 25° C.; and (b) a thickener in an amount which provides the composition to have a viscosity of from about 500 mPa·s to about 200,000 mPa·s, the thickener selected from the group consisting of fatty compounds, waxy compounds, gelling agents, inorganic thickeners, oil soluble polymers, and mixtures thereof.

### CROSS REFERENCE TO RELATED APPLICATION

The application is a continuation of International application PCT/US00/24018 (Case AA493F) filed on Aug. 31, 2000.

### FIELD OF THE INVENTION

The present invention relates to pre-shampoo conditioning compositions which deliver conditioning benefit to the hair by applying on dry hair and subsequently washing the hair with a shampoo. Specifically, the present invention relates to pre-shampoo conditioning compositions containing a conditioning oil and a thickener.

### BACKGROUND

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair”, or contribute to an undesirable phenomena of “split ends”, particularly for long hair.

A variety of approaches have been developed to alleviate these after-shampoo problems. These approaches range from post-shampoo application of hair conditioners such as leave-on and rinse-off products, to hair conditioning shampoos which attempt to both clean and condition the hair from a single product.

Although some consumers prefer the ease and convenience of a shampoo which includes conditioners, a substantial proportion of consumers prefer the more conventional conditioner formulations which are applied to the hair as a separate step from shampooing. Generally, there are two types of product forms that consumers prefer using. One form is the rinse off type conditioners, which would be applied during a bathing regimen subsequent to shampooing the hair, and typically be rinsed off from the hair. Another form is the leave on type conditioners, which would be applied to the hair in a dry or partially wet condition, typically as part of a styling regimen, and the hair would be left to dry.

The use on hair of oils which are liquid at room temperature, such as coconut oil, is known in the art. Such oils are difficult to effectively deliver to the hair. When such oils are contained in rinse off conditioning formulations and applied to the hair in a wet condition subsequent to shampooing, the oils would not deposit on and/or penetrate in the hair in an effective manner, and thus easily washed off in the rinsing process. When such oils are contained in leave on conditioning formulations and applied in a relatively dry condition and left to dry, the hair would be left with a sticky feel and a negative appearance. Thus, the conditioning benefit of oils were not achieved with the conventional method of use for such oils.

Based on the foregoing, there remains a desire to provide hair conditioning compositions which provide manageability and volume down to the hair while also providing a smooth, soft, and moisturized feel to the hair. There also remains a desire to provide hair conditioning compositions which provide such benefits without giving a sticky feel during and after use, or negative appearance after use.

None of the existing art provides all of the advantages and benefits of the present invention.

### SUMMARY

The present invention is directed to a hair conditioning composition for applying to the dry hair prior to washing the hair, comprising by weight:

(a) from about 30% to about 99.9% of a conditioning oil which is in liquid form at 25° C.; and

(b) a thickener in an amount which provides the composition to have a viscosity of from about 500 mPa·s to about 200,000 mPa·s, the thickener selected from the group consisting of fatty compounds, waxy compounds, gelling agents, inorganic thickeners, oil soluble polymers, and mixtures thereof.

These and other features, aspects, and advantages of the present invention will become evident to those skilled in the art from a reading of the present disclosure.

### DETAILED DESCRIPTION

While the specification concludes with claims which particularly point out and distinctly claim the invention, it is believed the present invention will be better understood from the following description.

All cited references are incorporated herein by reference in their entireties. Citation of any reference is not an admission regarding any determination as to its availability as prior art to the claimed invention.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Conditioning Oil

The composition of the present invention comprises by weight from about 30% to about 99.9%, preferably from about 50% to about 99.9% of a conditioning oil which is in liquid form at 25° C.

The conditioning oils useful herein are those which deposit and cover the surface of the hair when it is dry. Preferably, the conditioning oils useful herein are those which can, to some extent, penetrate into the hair and thus not easily washed off after subsequent shampooing.

Conditioning oils useful herein include those extracted or derived from a natural resource which contain a variety of compounds, and synthetic oils consisting of more or less a single compound. While volatile conditioning oils are not precluded herein, such oils are either kept to a relatively high boiling point or to a low level so as not to dry the hair, scalp, and hands.

Useful conditioning oils herein include vegetable oils, animal oils, and oils from other natural resources, synthetic oils, and mixtures thereof. The oils are selected according to the characteristics desired for the conditioner formulations. It is preferred that a certain amount of oils having a molecular weight of less than about 1500 is contained. Without being bound by theory, it is believed that oils having such molecular weight have the ability to penetrate into the hair.

In one preferred embodiment, vegetable oils comprising a high percentage of oleic acid and its triglyceride esters is preferred. Such high-oleic vegetable oils are believed to provide good penetration and coverage to the hair.

Vegetable oils useful herein are canola oil, camellia oil, olive oil, sunflower seed oil, cottonseed oil, soybean oil, peanut oil, olive oil, palm oil, corn oil, rapeseed oil, sesame oil, safflower oil, coconut oil, palm kernel oil, avocado oil, macadamia nut oil, corn oil, persic oil, wheat germ oil, pasanqua oil, linseed oil, perillic oil, teaseed oil, kaya oil, rice bran oil, china paulownia oil, Japanese paulownia oil, jojoba oil, rice germ oil, and mixtures thereof. Particularly preferred are canola oil, camellia oil, olive oil, sunflower seed oil, and mixtures thereof.

Animal oils and oils from other natural resources useful herein are sardine oil, lard, tallow, turtle oil, eggyolk oil, mink oil, squalane, lanolin, liquid lanolin, liquid paraffin, vaseline, and mixtures thereof.

Commercially available oils of natural resource useful herein include: canola oil with tradename Canola Salad Oil available from Ajinomoto, olive oil with tradename Fully Refined Olive Oil available from Hispanoliva, and sunflower seed oil with tradename Florasun 90 available from Floratech.

Fatty alcohols useful herein as a conditioning oil include those having from about 10 to about 30 carbon atoms, preferably from about 12 to about 22 carbon atoms, and more preferably from about 16 to about 22 carbon atoms. These fatty alcohols can be straight or branched chain alcohols and are preferably unsaturated alcohols. Nonlimiting examples of these compounds include oleyl alcohol, palmitoleic alcohol, linoleyl alcohol, and recinoleyl alcohol.

Fatty acids useful herein as a conditioning oil include those having from about 10 to about 30 carbon atoms, preferably from about 12 to about 22 carbon atoms, and more preferably from about 16 to about 22 carbon atoms. These fatty acids can be straight or branched chain acids and are unsaturated. Suitable fatty acids include, for example, oleic acid, linoleic acid, linolenic acid, ethyl linolenic acid, ethyl linolenic acid, arachidonic acid, and ricinolic acid.

Fatty acid derivatives and fatty alcohol derivatives useful herein as a conditioning oil are defined herein to include, for example, esters of fatty alcohols, alkoxylated fatty alcohols, alkyl ethers of fatty alcohols, alkyl ethers of alkoxylated fatty alcohols, and bulky ester oils such as pentaerythritol ester oils, trimethylol ester oils, citrate ester oils, glyceryl ester oils, and mixtures thereof. Nonlimiting examples of fatty acid derivatives and fatty alcohol derivatives, include, for example, methyl linoleate, ethyl linoleate, isopropyl linoleate, isodecyl oleate, isopropyl oleate, ethyl oleate, octyldodecyl oleate, oleyl oleate, decyl oleate, butyl oleate, methyl oleate, octyldodecyl stearate, octyldodecyl isostearate, octyldodecyl isopalmitate, octyl isopelargonate, octyl pelargonate, hexyl isostearate, isopropyl isostearate, isodecyl isononanoate, isopropyl isostearate, ethyl isostearate, methyl isostearate and Oleth-2. Bulky ester oils such as pentaerythritol ester oils, trimethylol ester oils, citrate ester oils and glyceryl ester oils useful herein are those which have a molecular weight of less than about 800, preferably less than about 500.

Hydrocarbons useful herein as a conditioning oil include straight chain, cyclic, and branched chain hydrocarbons which can be either saturated or unsaturated, so long as they have a melting point of not more than about 25° C. These hydrocarbons have from about 12 to about 40 carbon atoms, preferably from about 12 to about 30 carbon atoms, and preferably from about 12 to about 22 carbon atoms. Also encompassed herein are polymeric hydrocarbons of alkenyl monomers, such as polymers of C₂₋₆ alkenyl monomers. These polymers can be straight or branched chain polymers. The straight chain polymers will typically be relatively short in length, having a total number of carbon atoms as described above. The branched chain polymers can have substantially higher chain lengths. The number average molecular weight of such materials can vary widely, but will typically be up to about 500, preferably from about 200 to about 400, and more preferably from about 300 to about 350. Also useful herein are the various grades of mineral oils. Mineral oils are liquid mixtures of hydrocarbons that are obtained from petroleum. Specific examples of suitable hydrocarbon materials include paraffin oil, mineral oil, dodecane, isododecane, hexadecane, isohexadecane, eicosene, isoeicosene, tridecane, tetradecane, polybutene, polyisobutene, and mixtures thereof.

Commercially available fatty alcohols and their derivatives useful herein include: oleyl alcohol with tradename UNJECOL 90BHR available from Shin Nihon Rika, various liquid esters with tradenames SCHERCEMOL series available from Scher, and hexyl isostearate with a tradename HIS and isopropryl isostearate having a tradename ZPIS available from Kokyu Alcohol. Commercially available bulky ester oils useful herein include: trimethylolpropane tricaprylate/tricaprate with tradename MOBIL ESTER P43 from Mobil Chemical Co. Commercially available hydrocarbons useful herein include isododecane, isohexadeance, and isoeicosene with tradenames PERMETHYL 99A, PERMETHYL 101A, and PERMETHYL 1082, available from Presperse (South Plainfield N.J., USA), a copolymer of isobutene and normal butene with tradenames INDOPOL H-100 available from Amoco Chemicals (Chicago Ill., USA), mineral oil with tradename BENOL available from Witco, and isoparaffin with tradename ISOPAR from Exxon Chemical Co. (Houston Tex., USA).

Poly α-olefin oils useful herein are those derived from 1-alkene monomers having from about 6 to about 16 carbons, preferably from about 6 to about 12 carbons atoms. Nonlimiting examples of 1-alkene monomers useful for preparing the poly α-olefin oils include 1-hexene, 1-octene, 1-decene, 1-dodecene, 1-tetradecene, 1-hexadecene, branched isomers such as 4-methyl-1-pentene, and mixtures thereof. Preferred 1-alkene monomers useful for preparing the poly α-olefin oils are 1-octene, 1-decene, 1-dodecene, 1-tetradecene, 1-hexadecene, and mixtures thereof. Poly α-olefin oils useful herein further have a viscosity of from about 1 to about 35,000 cst, a molecular weight of from about 200 to about 60,000, preferably less than about 6,000, and more preferably less than about 800; and a polydispersity of no more than about 3.

Commercialy available poly α-olefin oils herein include polydecenes with tradenames PURESYN 6 having a number average molecular weight of about 500 available from Mobil Chemical Co.

High molecular weight ester oils useful herein as a conditioning oil include pentaerythritol ester oils, trimethylol ester oils, citrate ester oils, glyceryl ester oils, and mixtures thereof. The high molecular weight ester oils herein are “water-insoluble”. As used herein, the term “water-insoluble” means the compound is substantially not soluble in water at 25° C.; when the compound is mixed with water at a concentration by weight of above 1.0%, preferably at above 0.5%, the compound is temporarily dispersed to form an unstable colloid in water, then is quickly separated from water into two phases.

Pentaerythritol ester oils useful herein are those having the following formula:

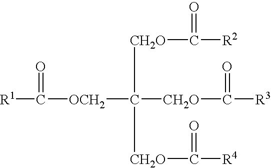

wherein R¹, R², R³, and R⁴, independently, are branched, straight, saturated, or unsaturated alkyl, aryl, and alkylaryl groups having from 1 to about 30 carbons. Preferably, R¹, R², R³, and R⁴, independently, are branched, straight, saturated, or unsaturated alkyl groups having from about 8 to about 22 carbons. More preferably, R¹, R², R³ and R⁴ are defined so that the molecular weight of the compound is from about 500 to about 1200.

Trimethylol ester oils useful herein are those having the following formula:

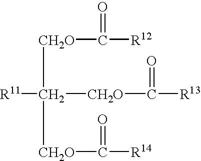

wherein R¹¹ is an alkyl group having from 1 to about 30 carbons, and R¹², R¹³, and R¹⁴, independently, are branched, straight, saturated, or unsaturated alkyl, aryl, and alkylaryl groups having from 1 to about 30 carbons. Preferably, R¹¹ is ethyl and R¹², R¹³, and R¹⁴, independently, are branched, straight, saturated, or unsaturated alkyl groups having from 8 to about 22 carbons. More preferably, R¹¹, R¹², R¹³ and R¹⁴ are defined so that the molecular weight of the compound is from about 500 to about 1200.

Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Citrate ester oils useful herein are those having a molecular weight of at least about 500 having the following formula:

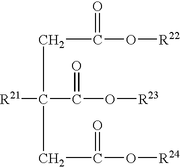

wherein R²¹ is OH or CH₃COO, and R²², R²³, and R²⁴, independently, are branched, straight, saturated, or unsaturated alkyl, aryl, and alkylaryl groups having from 1 to about 30 carbons. Preferably, R²¹ is OH, and R²², R²³, and R²⁴, independently, are branched, straight, saturated, or unsaturated alkyl, aryl, and alkylaryl groups having from 8 to about 22 carbons.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bernel.

Glyceryl ester oils useful herein are those having a molecular weight of at least about 500 and having the following formula:

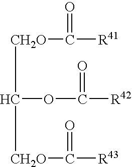

wherein R⁴¹, R⁴², and R⁴³, independently, are branched, straight, saturated, or unsaturated alkyl, aryl, and alkylaryl groups having from 1 to about 30 carbons. Preferably, R⁴¹, R⁴², and R⁴³, independently, are branched, straight, saturated, or unsaturated alkyl, aryl, and alkylaryl groups having from 8 to about 22 carbons.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Thickener

The composition of the present invention comprises a thickener in an amount which provides the composition to have a viscosity of from about 500 mPa·s to about 200,000 mPa·s, preferably from about 1,000 mPa·s to about 100,000 mPa·s. Such viscosity is suitable for providing a product which is easily applied to the hair, while not being too sticky. The viscosity herein is measured by a Brookfield DV-II+viscometer. When the viscosity is above about 10,000 mPa·s, a #52 spindle is used and measured at a condition of using 0.5 ml of the sample at 1 rpm after 1 minute at 27° C. When the viscosity is below about 10,000 mPa·s, a #41 spindle is used and measured at a condition of using 2 ml of the sample at 1 rpm after 3 minutes at 27° C.

The thickeners useful herein are selected from the group consisting of fatty compounds, waxy compounds, gelling agents, inorganic thickeners, oil soluble polymers, and mixtures thereof. The amount and type of thickeners are selected according to the desired viscosity and characteristics of the product. Preferably, a combination of thickeners are used. Preferably, the total amount of thickeners used is from about 0.1% to about 60%.

In one preferred embodiment, a thickener comprising a combination of from about 0.1% to about 10% fatty alcohol and from about 0.1% to about 50%, preferably up to about 30% of waxy compounds is used. In another preferred embodiment, from about 0.1% to about 10% of gelling agents is used. In another preferred embodiment, a thickener comprising from about 0.1% to about 10% inorganic thickeners is used.

Fatty Compounds

The fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that, depending on the number and position of double bonds, and length and position of the branches, certain compounds having certain required carbon atoms may have a melting point of less than 25° C. The fatty compounds useful as thickeners herein are only those having a melting point of 25° C. or higher.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago Ill., USA), HYSTRENE available from Witco Corp. (Dublin Ohio, USA), and DERMA available from Vevy (Genova, Italy).

Waxy Compounds

Waxy compounds are useful as thickeners of the present invention. Useful herein are petrolatum, which is a mixture of hydrocarbons obtained from petroleum and which is semisolid at room temperature, paraffin wax, microcrystalline wax, ozokerite wax, ceresin wax, carnauba wax, candellila wax, eicosanyl behenate, and mixtures thereof. Petrolatum is particularly preferred. Commercially available waxy compounds useful herein include petrolatum having a tradename Super White Protopet available from Witco, Candelilla wax NC-1630 available from Noda wax, Ozokerite wax SP-1021 available from Strahl & Pitsh, and Eicosanyl behenate available from Cas Chemical.

Gelling Agents

The gelling agents useful as thickeners of the present invention include esters and amides of fatty acid gellants, hydroxy acids, hydroxy fatty acids, cholesterolic materials, lanolinolic materials, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:

R²—NH—CO—(CH₂)₂—CH—(NH—CO—R¹)—CO—NH—R²

wherein R¹ is an aliphatic hydrocarbon radical having from about 12 to about 22 carbon atoms, and R² is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercial available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:

R¹—CO—(CH₂)₁₀—CH—(OH)—(CH₂)₅—CH₃

wherein R¹ is R² or NR²R³; and R² and R³ are hydrogen, or an alkyl, aryl, or arylalkyl radical which is branched linear or cyclic and has from about 1 to about 22 carbon atoms; preferably, from about 1 to about 18 carbon atoms. R² and R³ may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1 -propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Preferred solid non-polymeric gellants for use herein include those enantomeric compounds or materials containing at least one asymmetric (chiral) carbon atom. Non-limiting examples of these preferred enantomeric gellants include 1 2-hydroxystearic acid, other hydroxy acids such as alpha hydroxy acids, cholesterols, lanolin, and derivatives thereof.

Inorganic Thickeners

Inorganic thickeners useful herein include silica, oil soluble clays, and mixtures thereof. Highly dispersed, amorphous silicon dioxide of submicron particle size, also known as fumed silica, are particularly useful. Such material is commercially available as the Aerosil series (200, 300, 200CF, and 300CF) available from Degussa.

Oil Soluble Polymers

Oil soluble polymers are useful as thickeners of the present invention. Oil soluble polymers useful herein include guar gum which is a resinous material derived from the ground endosperm of cyanopsis tetragonoloba and close relatives.

Silicone Compound

Silicone compounds may be included in composition of the present invention to provide additional smooth feel when the hair is dried, and friction reduction.

The silicone compounds useful herein include volatile soluble or insoluble, or nonvolatile soluble or insoluble silicone conditioning agents. By soluble what is meant is that the silicone compound is miscible with the carrier of the composition so as to form part of the same phase. By insoluble what is meant is that the silicone forms a separate, discontinuous phase from the carrier, such as in the form of an emulsion or a suspension of droplets of the silicone. The silicone compounds herein may be made by any suitable method known in the art, including emulsion polymerization. The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

The silicone compounds for use herein will preferably have a viscosity of from about 1,000 to about 2,000,000 centistokes at 25° C., more preferably from about 10,000 to about 1,800,000, and even more preferably from about 100,000 to about 1,500,000. The viscosity can be measured by means of a glass capillary viscometer as set forth in Dow Corning Corporate Test Method CTM0004, Jul. 20, 1970. Silicone compound of high molecular weight may be made by emulsion polymerization. Suitable silicone fluids include polyalkyl siloxanes, polyaryl siloxanes, polyalkylaryl siloxanes, polyether siloxane copolymers, and mixtures thereof. Other nonvolatile silicone compounds having hair conditioning properties can also be used.

The silicone compounds herein also include polyalkyl or polyaryl siloxanes with the following structure (I)

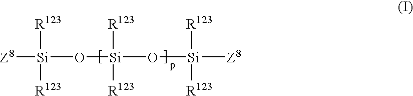

wherein R¹²³ is alkyl or aryl, and x is an integer from about 7 to about 8,000. Z⁸ represents groups which block the ends of the silicone chains. The alkyl or aryl groups substituted on the siloxane chain (R¹²³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R¹²³ groups on the silicon atom may represent the same group or different groups. Preferably, the two R¹²³ groups represent the same group. Suitable R¹²³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company (Waterford, N.Y., USA) in their Viscasil® and SF 96 series, and from Dow Corning Corp. (Midland, Mich., USA) in their Dow Corning 200 series and BY22-067.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Especially preferred, for enhancing the shine characteristics of hair, are highly arylated silicone compounds, such as highly phenylated polyethyl silicone having refractive index of about 1.46 or higher, especially about 1.52 or higher. When these high refractive index silicone compounds are used, they should be mixed with a spreading agent, such as a surfactant or a silicone resin, as described below to decrease the surface tension and enhance the film forming ability of the material.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispensability characteristics of the silicone. These material are also known as dimethicone copolyols.

Other silicone compounds include amino substituted materials. Suitable alkylamino substituted silicone compounds include those represented by the following structure (II)

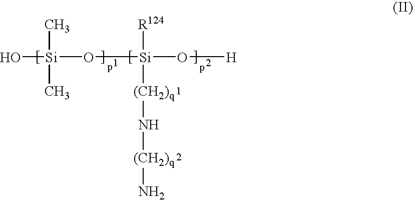

wherein R¹²⁴ is H, CH₃ or OH, p¹, p², q¹ and q² are integers which depend on the molecular weight, the weight average molecular weight being approximately between 5,000 and 10,000. This polymer is also known as “amodimethicone”. These Amodimethicones are available, for example, from Dow Corning as SM8704C.

Suitable amino substituted silicone fluids include those represented by the formula (III)

(R¹²⁵)ₐG₃₋ₐ—Si—(OSiG₂)ₚ₃—(OSiG_{b}(R¹²⁵)_{2-b})ₚ₄—O—SiG₃₋ₐ(R¹²⁵)ₐ (III)

in which G is chosen from the group consisting of hydrogen, phenyl, OH, C₁-C₈ alkyl and preferably methyl; a denotes 0 or an integer from 1 to 3, and preferably equals 0; b denotes 0 or 1 and preferably equals 1; the sum p³+p⁴ is a number from 1 to 2,000 and preferably from 50 to 150, p³ being able to denote a number from 0 to 1,999 and preferably from 49 to 149 and p⁴ being able to denote an integer from 1 to 2,000 and preferably from 1 to 10; R¹²⁵ is a monovalent radical of formula C_{q3}H_{2q3}L in which q³ is an integer from 2 to 8 and L is chosen from the groups

—N(R¹²⁶)CH₂—CH₂—N(R¹²⁶)₂

—N(R¹²⁶)₂

—N(R¹²⁶)₃X′

—N(R¹²⁶)CH₂—CH₂—NR¹²⁶H₂X′

in which R¹²⁶ is chosen from the group consisting of hydrogen, phenyl, benzyl, a saturated hydrocarbon radical, preferably an alkyl radical containing from 1 to 20 carbon atoms, and X′denotes a halide ion.

An especially preferred amino substituted silicone corresponding to formula (III) is the polymer known as “trimethylsilylamodimethicone” wherein R¹²⁴ is CH₃.

Other amino substituted silicone polymers useful herein include cationic amino substituted silicones represented by the formula (V):

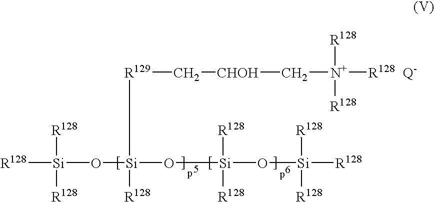

where R¹²⁸ denotes a monovalent hydrocarbon radical having from 1 to 18 carbon atoms, preferably an alkyl or alkenyl radical such as methyl; R¹²⁹ denotes a hydrocarbon radical, preferably a C₁-C₁₈ alkylene radical or a C₁-C₁₈, and more preferably C₁-C₁₈, alkyleneoxy radical; Q is a halide ion, preferably chloride; p⁵ denotes an average statistical value from 2 to 20, preferably from 2 to 8; p⁶ denotes an average statistical value from 20 to 200, and preferably from 20 to 50. A preferred polymer of this class is available from Union Carbide under the name “UCAR SILICONE ALE 56.”

References disclosing suitable nonvolatile dispersed silicone compounds include U.S. Pat. No. 2,826,551 to Geen; U.S. Pat. No. 3,964,500 to Drakoff, issued Jun. 22, 1976; U.S. Pat. No. 4,364,837 to Pader, issued Dec. 21, 1982; and British Pat. No. 849,433 to Woolston. “Silicon Compounds” distributed by Petrarch Systems, Inc., 1984, provides an extensive, though not exclusive, listing of suitable silicone compounds.

Another nonvolatile dispersed silicone that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416 to Spitzer, et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. The “silicone gums” will typically have a weight average molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. These silicone gums are typically provided as a blend with lower molecular weight silicone oils.

Also useful are silicone resins, which are highly crosslinked polymeric siloxane systems. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein.

Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone compounds on the hair and can enhance the glossiness of hair with high refractive index volumes.

Other useful silicone resins are silicone resin powders such as the material given the CTFA designation polymethylsilsequioxane, which is commercially available as Tospearl™ from Toshiba Silicones.

The method of manufacturing these silicone compounds, can be found in Encyclopedia of Polymer Science and Engineering, Volume 15, Second Edition, pp. 204-308, John Wiley & Sons, Inc., 1989.

Silicone materials and silicone resins in particular, can conveniently be identified according to a shorthand nomenclature system well known to those skilled in the art as the “MDTO” nomenclature. Under this system, the silicone is described according to the presence of various siloxane monomer units which make up the silicone. Briefly, the symbol M denotes the mono-functional unit (CH₃)₃SiO_{0.5}; D denotes the difunctional unit (CH₃)₂SiO; T denotes the trifunctional unit (CH₃)SiO_{1.5}; and Q denotes the quadri- or tetra-functional unit SiO2. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with the weight average molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the weight average molecular weight of the resin is from about 1000 to about 10,000.

Commercially available silicone compounds which are useful herein include Dimethicone with tradename D-130, cetyl Dimethicone with tradename DC2502, stearyl Dimethicone with tradename DC2503, emulsified polydimethyl siloxanes with tradenames DC1664 and DC1784, and alkyl grafted copolymer silicone emulsion with tradename DC2-2845; all available from Dow Corning Corporation (Midland, Mich., USA), emulsion polymerized Dimethiconol available from Toshiba Silicone Co., Ltd. (Tokyo, Japan) as described in GB application 2,303,857, mixture of Dimethicone and Dimethiconol with tradename DCQ2-1403, and mixture of Cyclomethicone and Dimethiconol with tradename DRQ2-1401, both mixtures available from Dow Corning.

Sensates

The hair conditioning composition of the present invention may further contain a sensate. As used herein the term “sensate” means a substance that, when applied to the skin, causes a perceived sensation of a change in conditions, for example, but not limited to, heating, cooling, refreshing and the like. Further, sensates may provide reduction of perceived oily and/or sticky feel during use. Still further, sensates may function as a preservative for the composition.

Sensates are preferably utilized at levels of from about 0.001% to about 10%, more preferably from about 0.005% to about 5%, even more preferably from about 0.01% to about 1%, by weight, of the total compositions.

Any sensate suitable for use in hair care compositions may be used herein. Preferred sensates for use in the compositions herein are camphor, menthol, I-isopulegol, ethyl menthane carboxamide and trimethyl isopropyl butanamide.

Additional Components

A wide variety of other additional components can be formulated into the present compositions. These include: other conditioning agents such as Salcare SC96 which is a mixture of Polyquaternium 37, propylene glycol dicaprylate dicaprate, and PPG-1 trideceth-6 commercially available from Ciba Specialty Chemicals, hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate, antidandruff agents such as zinc pyridinethione; and optical brighteners, for example polystyryistilbenes, triazinstilbenes, hydroxycoumarins, aminocoumarins, triazoles, pyrazolines, oxazoles, pyrenes, porphyrins, imidazoles, and mixtures thereof.

Composition and Method of Use

The conditioning compositions of the present invention are for use to apply on dry hair prior to washing the hair with a shampoo. It has been surprisingly found that the conditioning compositions deliver conditioning benefit to the hair such as manageability and volume down to the hair when used in such way. In the present invention, total hair volume is considered as the sum of the “flyaway volume” and the “bulk hair volume.” Accordingly, it has been found that reducing the bulk hair volume can therefore play a significant role in reducing the total hair volume. While certain compounds and compositions are known to reduce fly-away hair volume, for example, by reducing the static charge of hair, these compositions do not noticeably reduce bulk hair volume. It has now been found that when applied to hair, a hair care composition as described herein may reduce bulk hair volume, and may also reduce flyaway hair volume. This, in turn, provides a noticeable reduction in total hair volume.

A suitable method of using the present composition comprises the steps of;

(a) applying the hair conditioning composition according to any of the claims above to dry hair; and

(b) shampooing the hair with a shampoo composition comprising a detersive surfactant.

By applying the present composition to the dry hair, it is believed that the oils deposit and cover the surface of the hair, or to some extent, penetrate into the hair, and thus stay on the hair even during the washing process of the hair. Accordingly, it is an element of the method of the present invention to apply the present composition to dry hair, rather than after the hair is wetted with water. Without being bound by theory, it is believed that, by treating the hair prior to shampooing and then washing the hair, soils on the hair are removed, while a certain amount of the conditioning oils of the present invention are left on the hair. The conditioning oil left on the hair is believed to provide significantly better manageability and appearance benefits to the hair compared to when the hair is washed without pretreatment of the hair with the present composition.

The shampoo composition to be used in step (b) can be any composition comprising detersive surfactants and is suitable for washing off soils from the hair. The term detersive surfactant, as used herein, is intended to distinguish these surfactants from surfactants which are primarily emulsifying surfactants, i.e. surfactants which provide an emulsifying benefit and which have low cleansing performance. It is recognized that most surfactants have both detersive and emulsifying properties. It is not intended to exclude emulsifying surfactants from the present invention, provided the surfactant also possesses sufficient detersive properties to be useful herein. Detersive surfactants are typically selected from the group consisting of anionic surfactants, amphoteric surfactants, nonionic surfactants, and mixtures thereof. In one preferred embodiment, at least an anionic surfactant is included in the shampoo composition to be used in step (b).

The amount of the present composition used for treating the hair prior to shampooing depends on the volume of hair and desired conditioning benefits. Typically, from about 2 ml to about 40 ml, preferably from about 5 ml to about 20 ml of the present composition is used.

The present invention does not necessarily preclude overall hair treatment regimens wherein additional rinse off and/or leave on conditioning formulations are applied to the hair subsequent to washing the hair, or subsequent to washing and drying the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Examples 1 through 6 are hair conditioning compositions of the present invention which are used by applying to the dry hair prior to shampooing the hair.

Compositions of Examples 1 through 6

| Components | Ex. 1 | Ex. 2 | Ex. 3 | Ex 4 | Ex. 5 | Ex. 6 |
|---|---|---|---|---|---|---|
| Canola Oil *1 | 57.6 | | | | 57.5 | 58 |
| Olive Oil | | 96 | 99 | | | |
| Camellia Oil | | | | 93 | | |
| Petrolatum *2 | 30 | | | | 30 | 37 |
| Cetyl Alcohol *3 | 4.5 | | | 4.5 | 4.5 | |
| Stearyl Alcohol *4 | 2.5 | | | 2.5 | 2.5 | |
| Fumed silica *5 | | 4 | | | | |
| Dibutyl lauroyl glutamide *6 | | | 1 | | | |
| Silicone Blend *7 | 5 | | | | 5 | 5 |
| L-menthol | 0.1 | | | | | |
| Perfume | 0.3 | | | | | |
| Salcare SC96 *8 | | | | | 0.5 | |

Definitions of Components
*1 Canola Oil: Canola Salad Oil available from Ajinomoto
*2 Petrolatum: Super White Protopet available from Witco
*3 Cetyl Alcohol: Konol series available from Shin Nihon Rika.
*4 Stearyl Alcohol: Konol series available from Shin Nihon Rika.
*5 Fumed Silica: Aerosil 200CF series available from Degussa
*6 Dibutyl lauroyl glutamide: Gelling agent GP-1 available from Ajinomoto
*7 Silicone Blend: SE76 available from G.E.
*8 Salcare SC96: Mixture of Polyquaternium 37, propylene glycol dicaprylate dicaprate, and PPG-1 trideceth-6 available from Ciba Specialty Chemicals

Method of Preparation

The compositions of Examples 1 through 6 as shown above can be prepared by any conventional method as follows: Conditioning oils and thickeners are heated and agitated at about 80° C. The obtained mixture is cooled down to room temperature, silicone compounds are added, if present, and further agitated. Finally,,heat sensitive components such as sensates and perfume are added and mixed.

The embodiments disclosed and represented by the previous examples have many advantages. For example, they can provide conditioning benefit such as reduction of bulk hair volume; frizz control; smooth, soft, and moisturized feel to the hair, and shine to the hair.

It is understood that the examples and embodiments described herein are for illustrative purposes only and that various modifications or changes in light thereof will be suggested to one skilled in the art without departing from its spirit and scope.

What is claimed is:

1. A hair conditioning composition for applying to dry hair prior to washing the hair, comprising by weight:
(a) from about 30% to about 99.9% of a conditioning oil which is in liquid form at 25° C.; and
(b) a thickener in an amount which provides the composition with a viscosity of from about 500 mPa·s to about 200,000 mPa·s, the thickener selected from the group consisting of fatty compounds, waxy compounds, gelling agents, inorganic thickeners, oil soluble polymers, and mixtures thereof.

2. The hair conditioning composition according to claim 1 wherein the conditioning oil comprises, by weight of the composition, at least about 50% of a vegetable oil.

3. The hair conditioning composition according to claim 2 wherein the vegetable oil comprises at least about 50% of oleic acid and its triglyceride esters.

4. The hair conditioning composition according to claim 1 comprising from about 0.1% to about 60% of the thickener.

5. The hair conditioning composition according to claim 1 wherein the thickener comprises, by weight of the composition, from about 0.1% to about 10% of fatty compounds and from about 0.1% to about 50% of waxy compounds.

6. The hair conditioning composition according to claim 1 wherein the thickener comprises, by weight of the composition, from about 0.1% to about 10% of inorganic thickeners.

7. The hair conditioning composition according to claim 1 wherein the thickener comprises, by weight of the composition, from about 0.1% to about 10% of gelling agents.

8. The hair conditioning composition according to claim 1 further comprising a silicone compound.

9. The hair conditioning composition according to claim 1 further comprising a sensate.

10. A method of conditioning the hair comprising the steps of;
(a) applying the hair conditioning composition according to claim 1 to dry hair; and
(b) washing the hair with a shampoo composition comprising a detersive surfactant.

11. A method of reducing bulk hair volume of the hair comprising the steps of;
(a) applying the hair conditioning composition according to claim 1 to dry hair; and
(b) washing the hair with a shampoo composition comprising a detersive surfactant.

12. The method according to claim 10 wherein about 2 ml to about 40 ml of the hair conditioning composition is applied to the hair in step (a).
